(19) United States
(12) Patent Application Publication
(10) Pub. No.: US 2005/0131404 A1
(43) Pub. Date: Jun. 16, 2005

(54) Device for the connection between a shaft and a screw head with spherical symmetry

(76) Inventors: Keyvan Mazda, Paris (FR); Regis Le Couedic, Andresky (FR); Agnes Vaillant, Verneuil sur Seine (FR); Denis Pasquet, Aix En Provence (FR); Renaud Duchesnes, Diges (FR)

Correspondence Address: COHEN, PONTANI, LIEBERMAN & PAVANE, 551 FIFTH AVENUE, SUITE 1210, NEW YORK, NY, 10176, US

(21) Appl. No.: 10/504,409

(22) PCT Filed: Feb. 24, 2003
(86) PCT No.: PCT/FR03/00589

(30) Foreign Application Priority Data
Feb. 25, 2002 (FR) 02/02336

(51) Int. Cl. A61B 17/56

(52) U.S. Cl. 606/61

(57) ABSTRACT

The invention relates to a sequential connection device for connecting together a rod and a screw head having spherical symmetry. The device comprises a first longitudinal part and second longitudinal part suitable for co-operating together. Said first first end presenting a top portion and a curved bottom portion facing said top portion, said bottom portion presenting bottom bearing points against which said screw head can come to bear. Said top portion includes an adjustable thrust disposed facing said bottom portion and spaced apart from said screw head, the adjustable thrust and said bottom bearing point defining a polyhedron inside which the center C of said screw head can be situated.

The present invention relates to a sequential connection device for connecting together a rod and a spherically-symmetrical screw head, said rod and said screw head being situated close to each other.

The intended field of application is particularly, but not exclusively, that of orthopedics, and more particularly that of stabilizing the spine.

Stabilization devices are known that comprise pedicular screws inserted in the pedicles of vertebrae and at least one connection rod suitable for interconnecting said screws. The pedicular screws are inserted into the pedicles of successive vertebrae that are to be stabilized so that their heads project on either side of the spinous process. Thus, the screw heads on the same side of the spine can be interconnected by a rod for holding the vertebrae in positions that are fixed relative to one another.

To do this, the pedicular screws of said devices generally present a receiver head of U-shape suitable for receiving the rod, and the inside wall of the head is tapped on the axis of said screw so as to enable a locking part to be screwed against said rod. Thus, the rod is inserted into aligned receiver heads and is then secured to said heads by direct tightening of the locking parts which co-operate with the bottoms of the receiver heads in the manner of a vise.

Apart from the connection force between the rod and the screw head which is determined directly by the tightening force applied by the operator, which in turn depends on the operator's physical strength, the axis of the rod is necessarily situated in a plane that also contains the axis of the screw. Unfortunately, the positions of the screw heads are determined by the pedicles of the vertebrae in which the screws are engaged, and these positions are not the best positions for passing the rod, in particular in terms of optimizing compactness.

To solve this problem, devices for connecting together a screw head and a rod that are situated close to each other have been devised such that the rod can be placed between the row of spinous processes and the row of screw heads.

A problem which then arises and which the present invention seeks to solve is providing a device which makes it possible not only to hold the screw heads and the rod situated in the vicinity thereof firmly in fixed positions, but which also makes it possible during assembly to lock said rod partially relative to said heads, in particular to lock the direction in which it extends, while still allowing the rod to turn about its own axis and to move in translation along said axis.

To this end, in a first aspect, the device in accordance with the invention comprises: an elastically-deformable first longitudinal part extending between a first first end and a first second end and presenting an inside face, said first first end presenting a top portion and a curved bottom portion facing said top portion, said bottom portion presenting bottom bearing points against which said screw head can come to bear, said top portion including adjustable thrust means disposed facing said bottom portion and spaced apart from said screw head, the adjustable thrust means and said bottom bearing points defining a polyhedron inside which the center of said screw head can be situated; an elastically deformable, second longitudinal part suitable for co-operating with said first longitudinal part, which second part extends between a second first end and a second second end and presents a first face and a second face, said first face presenting intermediate bearing points at said second first end, said second first end of said second longitudinal part being suitable for being engaged between said screw head and said adjustable thrust means, said intermediate bearing points bearing against said screw head, said second second end being disposed facing said first second end, said rod being suitable for being placed between the inside face of the middle portion of the first longitudinal part and the middle portion of the second face of the second longitudinal part; and adjustable connection means for compressing said rod between the two middle portions; and firstly said adjustable thrust means are suitable for being actuated to bear against said second face at said second first end so as to compress said screw head between the bottom bearing points and the intermediate bearing points and to hold said longitudinal parts in a fixed position relative to each other and relative to said screw head, while the middle portions of the inside face and of the second face are held spaced apart from each other in a fixed position so as to allow said rod to move, and secondly said adjustable connection means are suitable for being actuated to hold said rod in a fixed position relative to said longitudinal parts.

Thus, a characteristic of the invention lies in the positions of the intermediate bearing points and of said bottom bearing points, which together define a polyhedron within which the center of said screw head can be situated. As a result, it will be understood that said bottom bearing points and the intermediate bearing points can form a vise for holding said second first end of said second longitudinal part in a fixed position against the spherical head and for locking together said head and said longitudinal parts without it being possible to disengage the spherically-symmetrical head. A sphere can be locked in position completely by four or more bearing points, providing the bearing points define a polyhedron containing the center of the sphere. In addition, the middle portions of the inside face and of the second face are held spaced apart from each other in a fixed position, thereby allowing said rod to be guided at least in translation along its longitudinal axis and at least in turning about said axis, while nevertheless being maintained in a single direction relative to said screw head. As a result, when devices in accordance with the invention are held in fixed positions relative to the screw heads and a rod is passing through them freely, the stress forces between the devices and the rod tend towards minimum values while said rod is being curved slightly for assembly reasons. Said rod then being movable to some extent relative to said devices.

Thereafter, the adjustable connection means in each of the devices can be actuated to lock said rod and said devices together. As explained in greater detail in the description below, it is by these adjustable connection means that secure locking of the rod relative to the screw heads is obtained.

In a particularly advantageous embodiment of the invention, said bottom portion presents a recess whose edge defines said bottom bearing points. Thus, according to this characteristic, the screw head can bear against the edge of the recess, while the shank of the screw passes through it so that the head is locked symmetrically on either side of the shank.

Advantageously, the edge of said recess forms at least three bottom bearing points, said three bearing points defining a triangle whose angles are all smaller than 90°, so that said screw head bearing against said three bearing points is prevented from moving in translation relative to said bottom portion in a direction parallel to the plane defined by said three bearing points. Thus, it will be understood that when the screw head is compressed against said three bearing points by a component perpendicular to the plane defined by said three bearing points, the screw head is prevented from moving in translation in a direction parallel to said plane.

In a preferred embodiment of the invention, said recess presents an edge defining at least a portion of a circle of peripheral length that is greater than the length of the periphery of the corresponding semicircle. As a result, the spherically-symmetrical screw head is also prevented from moving in translation in a direction parallel to the plane defined by said edge when the head is compressed perpendicularly to said plane.

In particularly advantageous manner, the surface of the top edge of said recess forms a portion of a truncated spherical ring suitable for co-operating with said screw head. Thus, the bottom bearing points form bearing surfaces of a shape that is complementary to the shape of the screw head, thereby providing a larger area of contact.

Preferably, the surface of the top edge of said recess forms a portion of a truncated frustoconical ring suitable for co-operating with said screw head so as to form a line of thrust between the spherical screw head and the frustoconical portion so as to enable locking to be firmer.

In a particularly advantageous embodiment of the invention, said intermediate bearing points form a surface defining at least a portion of a spherical cap suitable for co-operating with said screw head so as to present a large bearing surface against said screw head. It will thus be understood that the spherically-symmetrical screw head is securely engaged between said bottom portion and said second first end, and that forced displacement of one towards the other leads to both of them being locked about the screw head.

According to a particular advantageous characteristic, said first adjustable thrust means are constituted by a tapped hole passing right through said top portion of said first first end and screw fastener means suitable for being screwed into said tapped hole. As a result, the screw fastener means, which include an asymmetrical top socket into which driver means can be engaged, can be moved in translation towards said curved bottom portion.

Preferably, said second first end of said second longitudinal part presents a recess passing right through it and suitable for passing screw driver means suitable for being engaged in said spherically-symmetrical screw head in order to drive said screw.

In a preferred embodiment, said first adjustable thrust means and said second face at said second first end of said second longitudinal part against which they come to bear present abutment-forming means suitable for locking said longitudinal part relative to said first adjustable thrust means in a direction that is substantially perpendicular to the direction of the thrust force. As a result, said intermediate bearing means are securely held by said first adjustable thrust means and said screw head is locked more firmly between said bottom bearing means and said intermediate bearing points.

In this preferred embodiment, and advantageously, said screw fastener means present a thrust end presenting a shoulder which forms a projecting end, said shoulder being suitable for bearing against the perimeter of said recess in said second first end, said projecting end being suitable for being engaged in said recess of said second first end in order to form said abutment-forming means.

In a preferred embodiment of the invention, said adjustable connection means are formed by two tapped holes that are axially in line, formed respectively in the second ends of said longitudinal parts, and a screw having a threaded portion suitable for co-operating with said two tapped holes and an intermediate portion situated between said threaded portion and the head of the screw and suitable for being free relative to said tapped holes such that said threaded portion can co-operate with one only of the tapped holes in the two longitudinal parts and said head can co-operate with the other one of said longitudinal parts, so as to move said second ends towards each other, thereby compressing said rod between said middle portions. Thus, by moving the two second ends of the longitudinal parts towards each other, the rod is held captive between their middle portions, and is compressed so that the two longitudinal parts can be locked in a fixed position relative to the rod. Actuating the screw of the adjustable connection means serves firstly to move the longitudinal parts towards each other so that they bear against the rod, and then to apply compression to the rod between the two parts, which parts are suitable for bending.

In addition to clamping onto the rod, actuating the adjustable connection means enable the two longitudinal parts to pivot about the rod, thereby leading to additional compression of the spherically-symmetrical screw head between the curved bottom portion and the second first end of the second longitudinal part which are driven towards each other. As a result, it will be understood that the device in accordance with the invention is held in a fixed position on the spherically-symmetrical screw head in a manner that is more firm than that achieved by direct clamping between two vise-forming parts, since the lever effect produced by the rod situated between the two longitudinal parts enables the compression forces against the head to be increased.

In this preferred embodiment of the invention, the two second ends of the longitudinal parts are tapped so as to enable said screw to be secured to one of the two parts prior to connecting their ends together. In addition, because the intermediate portion of said clamping screw is free relative to the tapped holes, in particular in translation, the two second ends can be moved towards each other during tightening as soon as the tapped portion has left the tapped hole in the part against which the clamping head bears and has become engaged in the tapped hole in the other part. As explained in greater detail in the description below, the clamping screw is pre-screwed into the second end portion of the first longitudinal part in order, subsequently, to be screwed into the second second end of the longitudinal part.

In particularly advantageously manner, said middle portion of said inside face and said middle portion of said second face present semicylindrical recesses suitable for being placed facing each other in order to form a cylindrically-symmetrical housing in which said rod can be moved in rotation about its axis, and in translation along its axis. Thus, during assembly, the orientation of the rod can be held in a position that is fixed relative to the spherically-symmetrical head, while the rod is free to move in translation and in rotation relative to its longitudinal axis. As a result, the deformation exerted on the rod during assembly leads to minimizing the stresses on the devices through which the rod passes.

In another aspect, the present invention provides a kit for stabilizing the spine, the kit comprising: a plurality of pedicular screws each presenting a head with spherical symmetry; a plurality of sequential connection devices of the present invention; and at least one rod suitable for interconnecting said sequential connection devices.

Other features and advantages of the invention will appear on reading the following description of particular embodiments of the invention given by way of non-limiting indication, and with reference to the accompanying drawings, in which:

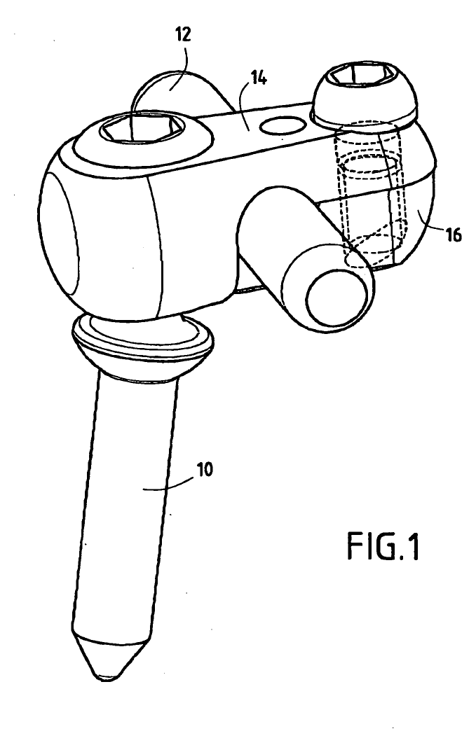

FIG. 1 is a diagrammatic perspective view of a device in accordance with the invention, shown on a screw and having a rod passing therethrough;

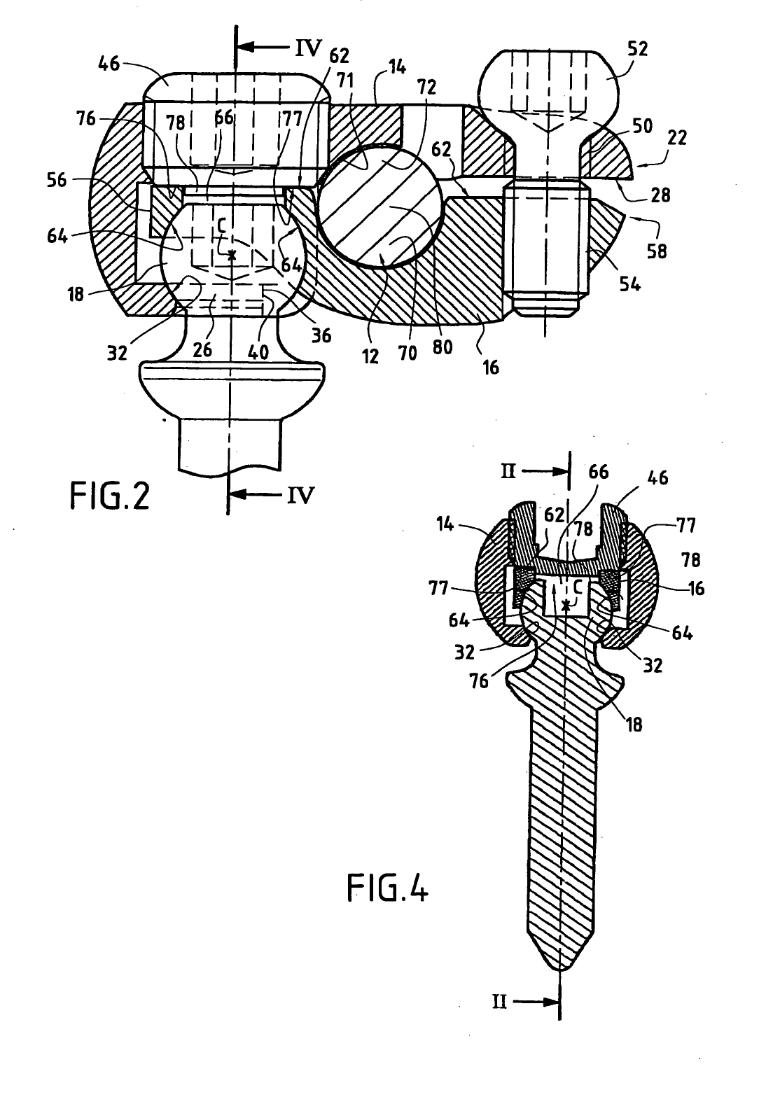

FIG. 2 is a diagrammatic longitudinal section view of the device shown in FIG. 1;

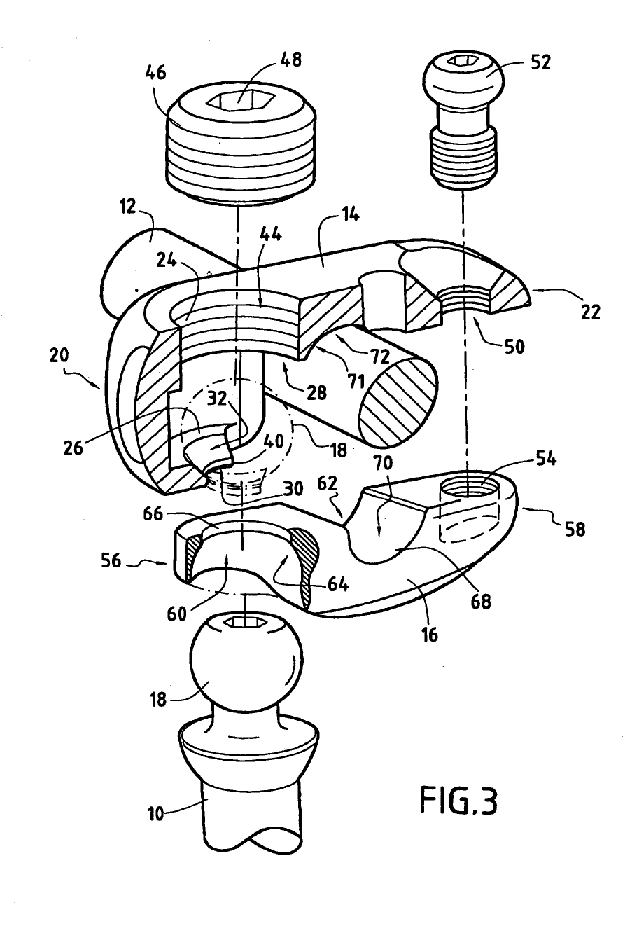

FIG. 3 is an exploded diagrammatic perspective view showing the device of the invention;

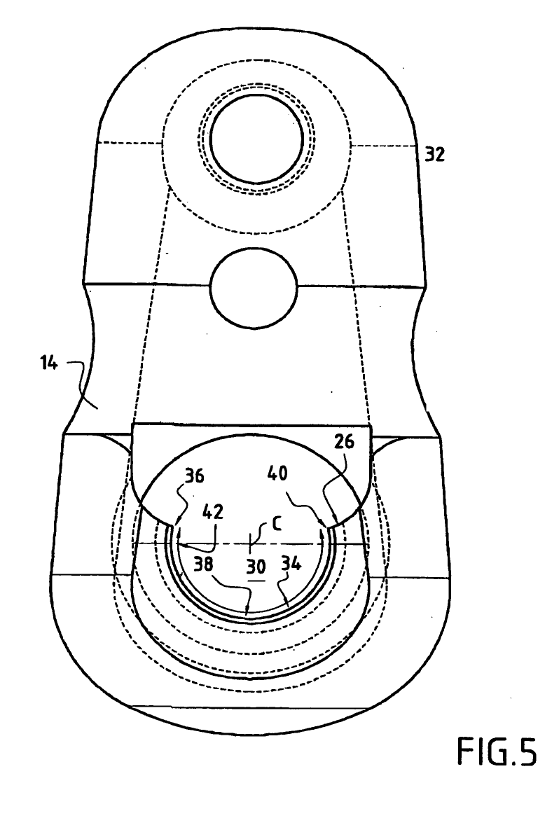

FIG. 4 is a diagrammatic cross-section view of the device shown in FIG. 1, in register with the screw; and

FIG. 5 is a diagrammatic view from beneath showing a part of said device.

FIG. 1 shows the device in accordance with the invention for connecting together a spherical head screw 10 and a rod 12 placed close to the screw 10. In this figure, there can be seen a first longitudinal part 14 and a second longitudinal part 16 which are described in greater detail in the description below. This type of screw is suitable for being inserted in the pedicle of a vertebra, and the heads of a plurality of screws are suitable for being connected together on the same side by a rod placed between the heads of the screws and the spinous processes of the vertebrae which project from the middles of the posterior walls of the vertebrae.

With reference to FIG. 3, there follows a description of the component elements of the sequential connection device in accordance with the invention. FIG. 3 shows the first longitudinal part 14 and the second longitudinal part 16 one above the other, together with the rod 12 and said screw 10 surmounted by a spherical head 18.

The first longitudinal part 14 has a first first end 20 and a first second end 22. The first first end 20 has a top portion 24 and a curved bottom portion 26 facing the top portion 24 and the inside face 28 of the first longitudinal part 14.

As shown in FIG. 3, the curved bottom portion 26 presents a recess 30 which can be seen in part only since the part is in longitudinal section in order to facilitate observation.

With reference to FIG. 5, there can be seen the first longitudinal part 14 as seen from beneath, and in particular the recess 30 whose top edge 32 (shown in FIG. 3), forms bottom bearing points against which the screw head 18 can come to bear.

The edge 34 of the recess 30, as shown in FIG. 5, forms three bottom bearing points 36, 38, and 40, which define a triangle whose angles are all less than 90° since the edge 34 of the recess 30 forms a portion of a circle 42 of peripheral length that is greater than the length of the periphery of the semicircle defined by said portion of a circle 42. In other words, the portion of a circle 42 has an angle at the center that is greater than 180°.

It will thus be understood that when the screw head 18 is engaged in the first longitudinal part 14, as represented by dashed lines in FIG. 3, so as to bear against the top edge 32, the portion of the spherically-symmetrical screw head 18 bearing against said top edge 32 defines a portion of a circle of circumference that is greater than the circumference of the semicircle defined by said portion of a circle. As a result, it will be understood that the spherical screw head 18 is held captive in translation in the plane defined by said portion of a circle, relative to the curved bottom portion 26 as shown in FIG. 5, the bearing points 36 and 40 being suitable for preventing it from moving, in particular in the direction going away from the bearing point 38.

The top edge 32 of the bottom portion 26 shown in FIG. 3 extends circularly between the bearing point 40 and the bearing point 36 which appears in FIG. 5 only. In a first embodiment, the surface of the top edge 32 forms a portion of a truncated spherical ring presenting a radius of curvature substantially equivalent to that of the spherical head 18 so as to match the shape thereof. The length of the periphery of the truncated spherical ring portion naturally corresponds to the length of the portion of a circle 42 shown in FIG. 5.

In a second embodiment, the surface of the top edge 32 forms a portion of a truncated frustoconical ring, so that the spherical head 18 comes into contact with said ring portion on a circular line, likewise corresponding to the portion of a circle 42. Thus, for equivalent pressure of the spherical head 18 against the top edge 32, a force per unit area is obtained that is greater, so the locking effect is improved.

As described in greater detail below, after describing the other characteristic elements of the invention which co-operate with the first longitudinal part 14 and the spherical head 18, this locking of the spherical head 18 against movement in translation is essential for achieving the object of the present invention.

The first longitudinal part 14, as shown in FIG. 3, has first adjustable bearing means formed by a tapped hole 44 passing right through the top portion 24, substantially perpendicularly to the longitudinal axis of the part 14 and directed towards the bottom portion 26; screw fastener means 46 are suitable for being screwed into the tapped hole 44. Thus, the fastener means 46 are suitable for being turned by means of a key suitable for co-operating with a hexagonal socket 48 formed in the top portion of the screw fastener means 46 in order to enable it to be driven in translation towards the bottom portion 26 through the space situated between the top portion 24 and the bottom portion 26.

In addition, the first second end 22 has a first tapped hole 50 in which a screw 52 is suitable for being screwed and which is suitable for co-operating with a second tapped hole 54 formed in the second longitudinal part 16 which is described in the description below, thereby forming adjustable connection means.

The second longitudinal part 16 presents a second first end 56 and a second second end 58, together with a first face 60 and a second face 62.

At the second first end, the first face 60 presents an intermediate bearing surface 64 having intermediate bearing points that can be seen in the cutaway portion of FIG. 3. The intermediate bearing surface 64, as shown, defines a surface forming a portion of a spherical cap, of curvature that is substantially identical to that of the spherical screw head 18 so as to form a large contact area between the spherical screw head 18 and the first face 60.

In addition, the second first end 56 presents a recess 66 passing right through it and opening out in register with the spherical screw head 18 when the head is engaged against the intermediate bearing surface 64. Where appropriate, this recess 66 makes it possible to insert means for driving the screw 10 into the spherical head 18, which head presents a hexagonal socket.

The middle portion 68 of the second face 62 of the second longitudinal part 16 presents a first semicylindrical recess 70 in which the rod 12 can come to bear; the middle portion 71 of the inside surface of the first longitudinal part 14 presents a second cylindrically-symmetrical recess 72 suitable for facing the semicylindrical recess 70.

With reference to FIGS. 2 and 4, there follows a description in greater detail of how the device in accordance with the invention operates and the stresses that act on the various parts while the device is mounted on the screw head 18 and has the rod 12 passing therethrough.

FIG. 2 shows all of the above-described parts, and in particular the first longitudinal part 14 having the second longitudinal part 16 placed facing its inside face 28. The inside face 28 and the second face 62 of the second longitudinal part 16 are disposed facing each other. The symmetrical semicylindrical recesses 70 and 72 are likewise placed facing each other so as to define a cylindrically-symmetrical housing 80 inside which the rod can initially be moved in translation and in rotation about its axis.

FIG. 2 shows even more clearly the essential means of the invention enabling the intended object to be achieved. Thus, the spherical screw head 18 presents a center C and it bears firstly against the top edge 32 which extends circularly on either side of the spherical screw head 18 as far as the bearing points 36 and 40 which are shown in dashed lines.

FIG. 4 shows the top edge 32 which extends on either side of the spherical screw head 18. On sight of FIGS. 2 and 4, it will be understood that the intermediate bearing points of the intermediate surface 64 of the second longitudinal part bear against the spherically-symmetrical screw head 18 and face the top edge 32 in such a manner that the intermediate bearing points of the intermediate bearing surface 64 co-operate with the bottom bearing points of the top edge 32 to define a polyhedron containing the center C of the spherical screw head 18.

In addition, the second first end 56 of the second longitudinal part 16 is disposed facing the adjustable bearing means, and in particular the screw fastener means 46 having an end 76 that bears against the second face 62 of the second longitudinal part 16, at the second first end 56.

The end 76 of the screw fastener means 46 presents a circular shoulder 77 forming a projecting portion 78 which is engaged in the recess 66 of the second first end 56 and which enables the second longitudinal part 16 to be prevented from moving in translation relative to the screw fastener means 46. The circular shoulder 77 bears against the perimeter of the recess 66.

Thus, turning the screw fastener means 46 drives the circular shoulder 77 in translation towards the bottom portion 26 against the perimeter of the recess 66 in the second first end 56. As a result, the intermediate bearing surface 64 of the second first end of the longitudinal part 16 bears against at least one top portion of the spherical surface of the spherical screw head 18. In addition, a bottom portion of the spherical surface of the spherical screw head 18 bears against the top edge 32 of the recess 30 in the bottom portion 26 and is prevented from moving in translation in the plane defined by the top edge 32.

Since the bearing points of the screw fastener means 46 and of the top edge 32 are being driven towards each other, and since they define at least one polyhedron containing the center C of the spherical screw head 18, and since the intermediate bearing points of the intermediate bearing structure 64 are held in a stationary position relative to the screw fastener means 46 and relative to the spherical screw head 18, and since they likewise define a polyhedron containing the center C, in association with the bottom bearing points of the top edge 32, tightening the screw fastener means 46 causes the first and second longitudinal parts 14 and 16 to be locked in a fixed position relative to each other and relative to the spherical screw head 18.

This locking of the spherical screw head 18 and of the two longitudinal parts 14, 16 is performed in such a manner that the head is completely secured to the assembly, and the two parts 14 and 16 are locked relative to each other without it being possible for the spherical screw head 18 to be detached. In addition, regardless of the angle at which the screw 10 is locked relative to the longitudinal parts 14, 16, the middle portions of these two parts are spaced apart from each other so that the rod 20 can still be free to move at least in rotation about its own axis and in translation along said axis.

Thus, the orientation of the rod 12 relative to the screw 10 can be maintained in a fixed position, with the rod 12 nevertheless retaining the ability to move relative thereto. This characteristic makes it possible to balance stresses between the rod and the plurality of devices that are suitable for being connected together.

The final stage of assembly consists in turning the screw 52 as shown in FIG. 2, which screw is initially pre-installed on the first longitudinal part 14 in the first tapped hole 50, prior to being screwed entirely into the second tapped hole 54.

The body of the screw 52 presents a non-threaded portion that is free to move in translation relative to the tapped hole and that is situated between its head and its threaded end so that the thread is released from the first tapped hole 50 when the screw is tightened, thereby causing the second ends 22 and 28 to move towards each other. Thus, the middle portions 68 and 71 are compressed towards each other and against the rod 12 so as to lock it in a position that is fixed relative to the longitudinal parts 14, 16, and thus relative to the spherical screw head 18.

When tightening the screw 52, and moving the second ends 22 and 58 towards each other, the longitudinal parts 14 and 16 deform, and in particular the first longitudinal part 14 bends so as to curve about the rod 12. In addition, moving the second ends 22 and 58 towards each other causes the longitudinal parts to pivot about the rod 12 and causes the second first end 56 to move towards the bottom portion 56, thereby clamping the spherical screw head more tightly between the first ends of the two longitudinal parts 14 and 16.

This terminal stage of assembly can be finished off by re-tightening the screw fastener means 46 since the stresses they exert on the first second end 56 might be reduced when the screw 52 is tightened since it tends to cause the two longitudinal parts 14 and 16 to pivot relative to each other.

The longitudinal parts 14 and 16 are preferably made of titanium alloy, so that the amount of deformation to which they are subjected under stress is small. Thus, tightening the screw 52 which serves in particular to cause the part 14 to bend, serves to guarantee a completely rigid connection between the spherical screw head 18 and the rod 12. Advantageously, certain stainless steels are suitable for being used.

The present invention also provides a kit for stabilizing the spine and suitable for being installed on the vertebral column of a patient. This kit comprises a plurality of pedicular screws having spherical heads suitable for being screwed in pairs into the two pedicles of a plurality of successive vertebrae so as to form two rows of screw heads on either side of the spinous processes. The kit also comprises a plurality of sequential connection devices in accordance with the invention for mounting on respective spherical screw heads, and two rods enabling the devices in each of the rows of screw heads to be connected together.

1-14. (canceled)

15. A sequential connection device for connecting together a rod and a spherically-symmetrical screw head, said rod and said screw head being situated close to each other,
the device comprising:
an elastically-deformable first longitudinal part extending between a first first end and a first second end and presenting an inside face, said first first end presenting a top portion and a curved bottom portion facing said top portion, said bottom portion presenting bottom bearing points against which said screw head can come to bear, said top portion including adjustable thrust means disposed facing said bottom portion and spaced apart from said screw head, the adjustable thrust means and said bottom bearing points defining a polyhedron inside which the center C of said screw head can be situated;
an elastically deformable, second longitudinal part suitable for co-operating with said first longitudinal part, said second part extending between the second first end and a second second end and presents a first face and a second face, said first face presenting intermediate bearing points at said second first end, said second first end of said second longitudinal part being suitable for being engaged between said screw head and said adjustable thrust means, said intermediate bearing points bearing against said screw head, said second second end being disposed facing said first second end, said rod being suitable for being placed between the inside face of the middle portion of the first longitudinal part and the middle portion of the second face of the second longitudinal part; and
adjustable connection means for compressing said rod between said two middle portions, wherein firstly said adjustable thrust means are suitable for being actuated to bear against said second face at said second first end so as to compress said screw head between said bottom bearing points and said intermediate bearing points and to hold said longitudinal parts in a fixed position relative to each other and relative to said screw head, while the middle portions of the inside face and of the second face are held spaced apart from each other in a fixed position to allow said rod to move, and secondly said adjustable connection means are suitable for being actuated to hold said rod in a fixed position relative to said longitudinal parts.

16. A sequential connection device according to claim 15, wherein said bottom portion has a recess with an edge, said edge defining said bottom bearing points.

17. A sequential connection device according to claim 16, wherein said edge of said recess forms at least three bottom bearing points, said three bottom bearing points defining a triangle whose angles are all smaller than 90°, whereby said screw head bearing against said three bottom bearing points is prevented from moving in translation relative to said bottom portion in a direction parallel to the plane defined by said three bottom bearing points.

18. A sequential connection device according to claim 17, wherein said recess has an edge defining at least a portion of a circle of peripheral length that is greater than the length of the periphery of the corresponding semicircle.

19. A sequential connection device according to claim 18, wherein the surface of the top edge of said recess forms a portion of a truncated spherical ring for co-operating with said screw head.

20. A sequential connection device according to claim 18, wherein the surface of the top edge of said recess forms a portion of a truncated frustoconical ring for co-operating with said screw head.

21. A sequential connection device according to claim 15, wherein said intermediate bearing points form a surface defining at least a portion of a spherical cap for co-operating with said screw head.

22. A sequential connection device according to claim 15 wherein said first adjustable thrust means comprise a tapped hole passing right through said top portion of said first first end and screw fastener means suitable for being screwed into said tapped hole.

23. A sequential connection device according to claim 22, wherein said second first end of said second longitudinal part has a recess passing right through it for passing screw driver means.

24. A sequential connection device according to claim 23, wherein said first adjustable thrust means and said second face at said second first end of said second longitudinal part against which they come to bear present abutment-forming means for locking said longitudinal part relative to said first adjustable thrust means in a direction that is substantially perpendicular to the direction of the thrust force.

25. A sequential connection device according to claim 15, wherein said second first end of said second longitudinal part has a recess passing right through it for passing screw driver means.

26. A sequential connection device according to claim 15, wherein said first adjustable thrust means and said second face at said second first end of said second longitudinal part against which they come to bear present abutment-forming means suitable ]for locking said longitudinal part relative to said first adjustable thrust means in a direction that is substantially perpendicular to the direction of the thrust force.

27. A sequential connection device according to claim 15, wherein said adjustable connection means comprise two tapped holes that are axially in line, formed respectively in the second ends of said longitudinal parts, and a screw having a threaded portion suitable for co-operating with said two tapped holes and an intermediate portion situated between said threaded portion and the head of the screw and suitable for being free relative to said tapped holes such that said threaded portion can co-operate with one only of the tapped holes in the two longitudinal parts and said head can co-operate with the other one of said longitudinal parts, so as to move said second ends towards each other, thereby compressing said rod between said middle portions.

28. A sequential connection device according to claim 27, wherein said screw fastener means present a thrust end having a shoulder which forms a projecting end, said shoulder being suitable for bearing against the perimeter of said recess in said second first end, said projecting end being suitable for being engaged in said recess of said second first end in order to form said abutment-forming means.

29. A sequential connection device according to claim 15, wherein said middle portion of said inside face and said middle portion of said second face have semicylindrical recesses suitable for being placed facing each other in order to form a cylindrically-symmetrical housing in which said rod can be moved in rotation about its axis, and in translation along its axis.

30. A kit for stabilizing the spine comprising:
a plurality of pedicular screws each presenting a head with spherical symmetry;
a plurality of sequential connection devices, each device comprising
an elastically-deformable first longitudinal part extending between a first first end and a first second end and presenting an inside face, said first first end presenting a top portion and a curved bottom portion facing said top portion, said bottom portion presenting bottom bearing points against which said screw head can come to bear, said top portion including adjustable thrust means disposed facing said bottom portion and spaced apart from said screw head, the adjustable thrust means and said bottom bearing points defining a polyhedron inside which the center C of said screw head can be situated;
an elastically deformable, second longitudinal part suitable for co-operating with said first longitudinal part, said second part extending between a second first end and a second second end and presents a first face and a second face, said first face presenting intermediate bearing points at said second first end, said second first end of said second longitudinal part being suitable for being engaged between said screw head and said adjustable thrust means, said intermediate bearing points bearing against said screw head, said second second end being disposed facing said first second end, said rod being suitable for being placed between the inside face of the middle portion of the first longitudinal part and the middle portion of the second face of the second longitudinal part:
adjustable connection means for compressing said rod between said two middle portions;
wherein firstly said adjustable thrust means are suitable for being actuated to bear against said second face at said second first end so as to compress said screw head between said bottom bearing points and said intermediate bearing points and to hold said longitudinal parts in a fixed position relative to each other and relative to said screw head, while the middle portions of the inside face and of the second face are held spaced apart from each other in a fixed position to allow said rod to move, and secondly said adjustable connection means are suitable for being actuated to hold said rod in a fixed position relative to said longitudinal parts; and
at least one rod suitable for interconnecting said sequential connection devices.
